(19) United States
(12) Patent Application Publication
(10) Pub. No.: US 2007/0049836 A1
(43) Pub. Date: Mar. 1, 2007

(54) Electronic wristwatch-type exercise signal detecting apparatus

(76) Inventor: Yu-Yu Chen, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/589,824

(22) Filed: Oct. 31, 2006

Related U.S. Application Data
(63) Continuation-in-part of application No. 11/004,977, filed on Dec. 7, 2004.

(51) Int. Cl. A61B 5/04

(52) U.S. Cl. 600/519

(57) ABSTRACT

An electronic wristwatch-type exercise signal detecting apparatus includes a watchcase having a pair of electronic vibration detectors provided therein for detecting a vibrating signal from a user in taking exercise. The detected vibrating signal is sent via a vibrating signal detecting circuit to a micro-controller in a control circuit, so that a numerical value representing the vibrating signal received by the micro-controller is shown on a display unit on the watchcase. An acceleration detector is further mounted in the watchcase for detecting an accelerating signal from the user in taking exercise. Moreover, a pair of electrically conductive contact areas are arranged at a top surface and a least one electrically conductive contact areas are arranged at the bottom surface of the watchcase for detecting the user's heartbeat signal.

### CROSS-REFERENCES TO RELATED APPLICATIONS

This application is a continuation-in-part of Ser. No. 11/004,977 filed Dec. 7, 2004 entitled “Electronic Wristwatch-Type Exercise Signal Detecting Apparatus”.

### FIELD OF THE INVENTION

The present invention relates to an electronic wristwatch-type exercise signal detecting apparatus, and more particularly to a wristwatch-type exercise signal detecting apparatus for detecting a user's body signals that are generated when the user is taking exercise.

### BACKGROUND OF THE INVENTION

Various types of sporting instruments have been developed for people who live in the busy modern society but pay more and more attention to proper exercises that are helpful to their health. Meanwhile, there are also various kinds of body signal sensing devices being developed for exercisers to understand and accurately control their physical conditions during taking exercise.

Among different exercise signal sensing devices, the pedometer has the advantages of being conveniently portable, easily operable, and available for measuring steps of walking or running, and is therefore widely welcome and adopted among consumers. The pedometer may be differently designed for attaching to a user's shoe, wearing on a user's waist, or wearing on a user's wrist like a wristwatch. The acceleration detector is another type of exercise signal sensing devices that is frequently used to detect the user's acceleration during exercising, so that the user may further evaluate his or her capacity of movement. In addition, the heartbeat/pulse detector is also frequently used to detect the user's heartbeats or pulses when the user is taking exercise, so as to help the user to understand and control his or her real physical conditions.

Most of the currently available pedometers typically include a mechanical vibration-detecting element, which uses a swinging element to touch a switch and thereby transmits a signal, so that a numerical value representing the number of steps is shown on a display. The conventional vibration-detecting element tends to have wrong motions after being used over a prolonged time, and must have a weight that must vibrate sufficiently to correctly count the number of steps. Generally speaking, the conventional vibration detecting element has a relatively low sensitivity. There are also some other conventional vibration detecting elements that include a magnetic reed switch. In this type of vibration detecting elements, there is included a swing arm that has a magnetic element attached thereto and counts the number of steps without contacting the switch. The magnetic reed switch requires high manufacturing cost and tends to be affected by nearby magnetic fields.

There are many patents of prior art disclosing different mechanical vibration detecting units. U.S. Pat. No. 4,460,823 discloses a pedometer for detecting a user's steps in walking or running. The pedometer includes a swinging weight, elastic elements, gears, etc., and a counter that indicates the number of steps when a user is walking or running. U.S. Pat. No. 4,560,861 discloses a pedometer that uses a swinging weight, elastic elements, and the like to measure the number of steps of the user in moving. U.S. Pat. No. 5,117,444 discloses a pedometer that uses a swinging weight, magnetic elements, a magnetic reed switch, and the like to measure the number of steps of the user in moving.

All the above-mentioned exercise signal detecting devices of prior art have the disadvantages of having complicate structure, requiring high manufacturing cost, producing noises during operation, having mechanical members that tend to become fatigued or have a reduced sensitivity after being used over a prolonged time. It is therefore desirable to improve the conventional mechanical exercise signal detecting devices.

### SUMMARY OF THE INVENTION

A primary object of the present invention is to provide an electronic wristwatch-type exercise signal detecting apparatus, which includes electronic exercise signal detectors capable of generating electronic signals to detect exercise signals from an exerciser, so as to overcome the drawbacks existed in the conventional mechanical exercise signal detecting devices.

Another object of the present invention is to provide a detecting apparatus that is able to detect vibrating signals, accelerating signals, and heartbeat/pulse signals from a user in taking exercise, so that the user could accurately understand and control his or her capacity of movement and real physical conditions during exercising.

A further object of the present invention is to provide an electronic wristwatch-type exercise signal detecting apparatus including two electronic exercise signal detectors. The first electronic exercise signal detector is oriented in X direction, while the second electronic exercise signal detector is oriented in a direction with an obtuse angle to the first electronic vibration detector. With this arrangement, even slow walking and light motion of the user can be detected precisely by the electronic wristwatch-type exercise signal detecting apparatus.

To achieve the above objects, in accordance with the present invention, there is provided an electronic wristwatch-type exercise signal detecting apparatus which include a watchcase having an electronic vibration detector provided therein for detecting a vibrating signal from a user in taking exercise. The detected vibrating signal is sent via a vibrating signal detecting circuit to a micro-controller in a control circuit, so that a numerical value representing the vibrating signal received by the micro-controller is shown on a display unit on the watchcase. An acceleration detector is further mounted in the watchcase for detecting an accelerating signal from the user in taking exercise. Moreover, a pair of electrically conductive contact areas are arranged at a bottom of the watchcase for detecting the user's heartbeat signal.

With the electronic vibration detector, the wristwatch-type exercise signal detecting apparatus of the present invention worn on a user's wrist is able to detect vibrating signals from the user in taking exercise, so as to provides the user with a numerical value as a reference. The electronic exercise signal detector of the present invention effectively overcomes the disadvantages of complicate structure, high manufacturing cost, operating noises, fatigued members and reduced mechanical sensitivity over a prolonged time, etc., as existed in the conventional mechanical vibration detecting devices.

The electronic vibration detector may be otherwise an acceleration detector for detecting accelerating signals from an exerciser in taking exercise.

In the present invention, numerical values representing the exercisers' heartbeat/pulse signals, the vibrating signals, and the accelerating signals may be shown on a display unit and stored in a memory for recording and analyzing the user's capacity of movement.

The structure and the technical means adopted by the present invention to achieve the above and other objects can be best understood by referring to the following detailed description of the preferred embodiments and the accompanying drawings, wherein

### BRIEF DESCRIPTION OF THE DRAWINGS

FIG. 1 is a perspective view of an electronic wristwatch-type exercise signal detecting apparatus according to the present invention;

FIG. 2 is a top plan view of the electronic wristwatch-type exercise signal detecting apparatus of FIG. 1;

FIG. 3 is a bottom plan view of the electronic wristwatch-type exercise signal detecting apparatus of FIG. 1;

FIG. 4 schematically shows the arrangement of related components inside the electronic wristwatch-type exercise signal detecting apparatus of the present invention;

FIG. 5 is a block diagram showing a control circuit for the electronic wristwatch-type exercise signal detecting apparatus of the present invention;

FIG. 6 is a waveform diagram showing the output voltage of a conventional vibration detector when a user jog or walk slowly; and

FIG. 7 is a waveform diagram showing the output voltage of the electronic wristwatch-type exercise signal detecting apparatus of the present invention when a user jog or walk slowly.

### DETAILED DESCRIPTION OF THE PREFERRED EMBODIMENTS

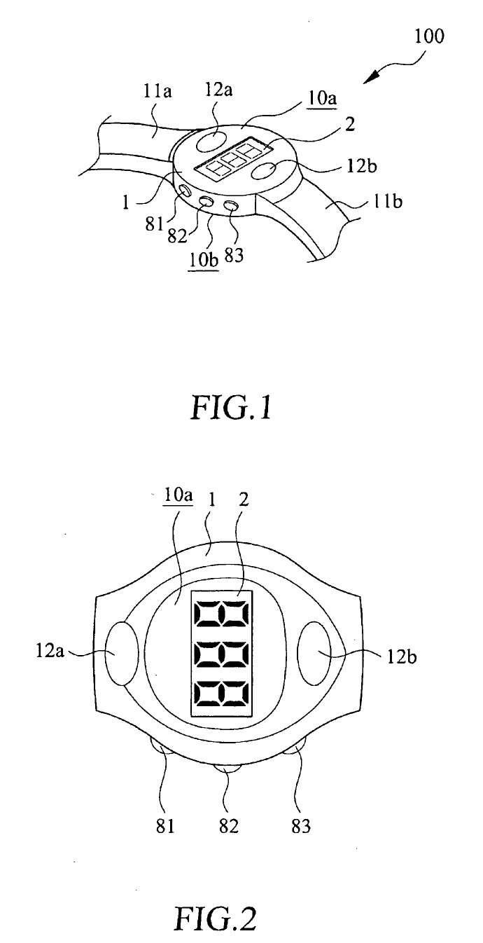

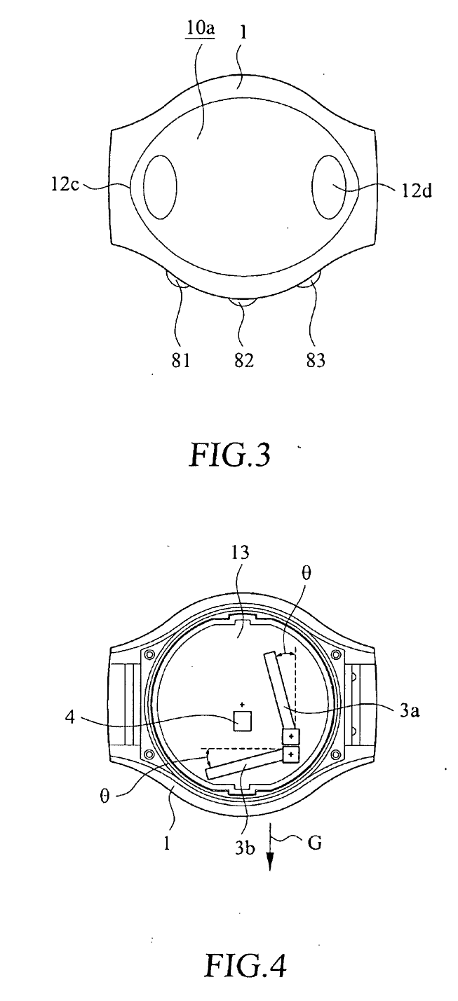

Please refer to FIGS. 1 to 3. FIG. 1 is a perspective view of an electronic wristwatch-type exercise signal detecting apparatus 100 according to the present invention, FIG. 2 is a top plan view and FIG. 3 is a bottom plan view of FIG. 1. As shown, the electronic wristwatch-type exercise signal detecting apparatus 100 includes a watchcase 1 with a top surface 10a and a bottom surface 10b. Two watchbands 11a, 11b are separately connected to two opposite ends of the watchcase 1 to enable wearing of the apparatus 100 on a user's wrist. A display unit 2 is provided on the top surface 10a of the watchcase 1.

Moreover, a pair of electrically conductive contact areas 12a, 12b are oppositely provided at the top surface 10a of the watchcase 1 and a pair of electrically conductive contact areas 12c, 12d are oppositely provided at the bottom surface 10b of the watchcase 1. When the electronic wristwatch-type exercise signal detecting apparatus 100 is worn on the user's wrist, the electrically conductive contact areas 12c, 12d are in contact with the user's skin of his left hand.

The electronic wristwatch-type exercise signal detecting apparatus 100 applies the technique of EKG for detection of heartbeat. Accordingly, when the user puts two fingers of his right hand on the electrically conductive contact areas 12a, 12b at the top surface 10a of the electronic wristwatch-type exercise signal detecting apparatus 100 which is in contact with the user's wrist through the conductive contact areas 12c, 12d at the bottom surface 10b, the circuits inside the watchcase 1 detect the user's heartbeat signals which are then shown on the display unit 2. In another embodiment, only one electrically conductive contact area 12c is mounted at the bottom surface 10b of the electronic wristwatch-type exercise signal detecting apparatus 100 and is capable to work with the electrically conductive contact areas 12a, 12b at the top surface 10a to detect the user's heartbeat signals.

Please refer to FIG. 4 that shows the arrangement of related components inside the electronic wristwatch-type exercise signal detecting apparatus 100. As shown, a base board 13 is provided in the watchcase 1, and a pair of electronic vibration detectors, the first electronic vibration detector 3a and the second electronic vibration detector 3b, and an acceleration detector 4 are provided on one surface of the base board 13.

The electronic vibration detectors 3a, 3b are adapted to generate an electronic signal respectively according to the user's body vibrating state during taking exercise, such as running, walking, etc. That is, whenever an exerciser wearing the apparatus 100 of the present invention moves one step during running or walking, each of the electronic vibration detectors 3a, 3b detects the vibrating state and generates a voltage variation. The detected voltage variation is then sent to a control circuit arranged on the base board 13 of the apparatus 100.

The electronic vibration detectors 3a, 3b are made of piezoelectric ceramic. The first electronic vibration detector 3a is mounted along a direction with an angle θ with respect to Y direction which is parallel to the gravitational force direction G of the gravitational field, while the second electronic vibration detector 3b is mounted along a direction with an angle θ with respect to the X direction which is parallel to the horizontal direction, when the electronic wristwatch-type exercise signal detecting apparatus 100 is put on the wrist of the user. Preferably, the first electronic vibration detector 3a is positioned with an angle θ in the range of 5 degrees to 30 degrees with respect to the gravitational force direction G of the gravitational field, and the second electronic vibration detector 3b is positioned with an angle θ in the range of 5 degrees to 30 degrees with respect to the X direction. The detection of vibration signals will be described in details below.

The acceleration detector 4 may be a single-axis, a two-axis, or a three-axis acceleration detector for detecting accelerating signals from a user in taking exercise.

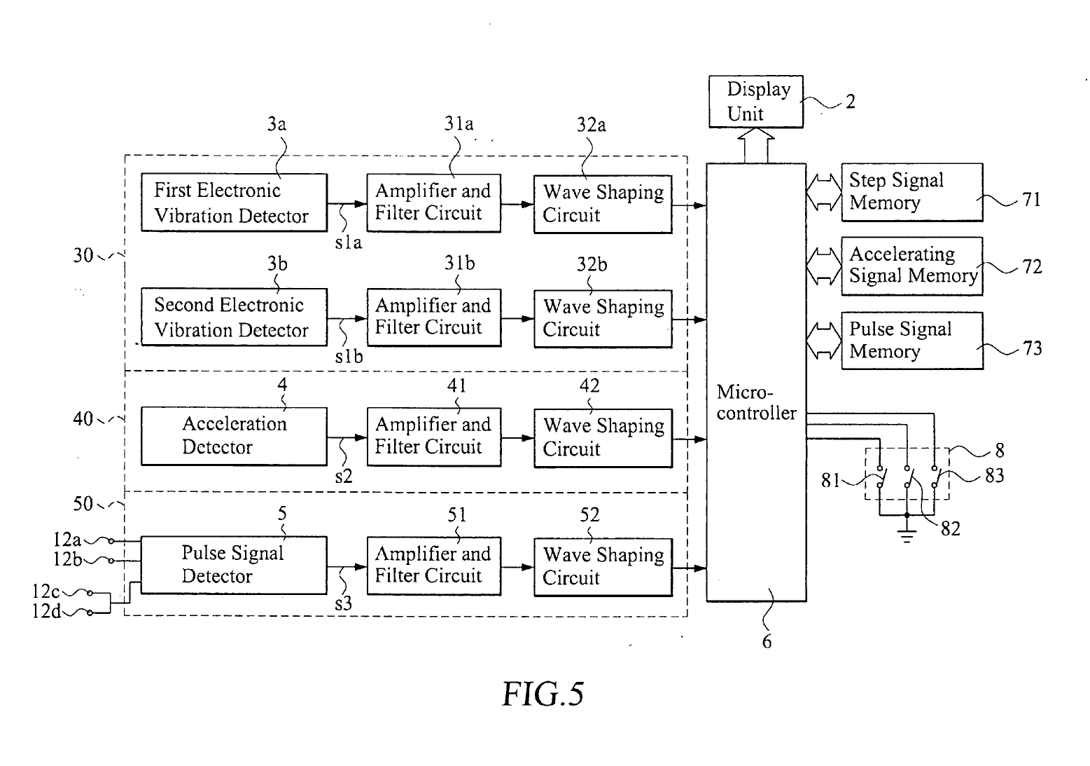

FIG. 5 is a block diagram showing a control circuit of the electronic wristwatch-type exercise signal detecting apparatus 100 of the present invention. As shown, the control circuit mainly includes a vibrating signal detecting circuit 30, an accelerating signal detecting circuit 40, and a pulse signal detecting circuit 50.

The vibrating signal circuit 30 includes the electronic vibration detectors 3a, 3b. The first electronic vibration detectors 3a is capable of generating a first vibrating signal s1a, which is amplified and subjected to noise filtering at an amplifier and filter circuit 31a before being sent to a wave shaping circuit 32a for signal shaping, and is finally sent to a micro-controller 6. The second electronic vibration detectors 3b is capable of generating a second vibrating signal s1b, which is amplified and subjected to noise filtering at an amplifier and filter circuit 31b before being sent to a wave shaping circuit 32b for signal shaping, and is finally sent to the micro-controller 6. The micro-controller 6 receives the vibrating signals s1a and s1b and then selectively converts the vibrating signal s1a or s1b into step signals dependent on the larger signal amplitude selected from the vibrating signal s1a or s1b. The step signals are then shown on the display unit 2. Alternatively, the step signals may be recorded and stored in a step signal memory 71.

The accelerating signal detecting circuit 40 includes the acceleration detector 4 for detecting an accelerating signal s2, which is amplified and subjected to noise filtering at an amplifier and filter circuit 41 before being sent to a wave shaping circuit 42 for signal shaping, and is finally sent to the micro-controller 6. The micro-controller 6 then displays the received accelerating signal s2 on the display unit 2. Alternatively, the accelerating signal may be recorded and stored in am accelerating signal memory 72.

The pulse signal detecting circuit 50 includes a pulse signal detector 5 for detecting a user's pulse signal s3 via the electrically conductive contact areas 12a, 12b, 12c, 12d. The detected pulse signal s3 is amplified and subjected to noise filtering at an amplifier and filter circuit 51 before being sent to a wave shaping circuit 52 for signal shaping, and is finally sent to the micro-controller 6. The micro-controller 6 then displays the received pulse signal s3 on the display unit 2. Alternatively, the pulse signal may be recorded and stored in a pulse signal memory 73.

The micro-controller 6 is electrically connected to a set of operating keys 8, which may, for example, include an on/off key 81, a mode-selection key 82, and a clear key 83, for the user to control the electronic wristwatch-type exercise signal detecting apparatus 100 of the present invention.

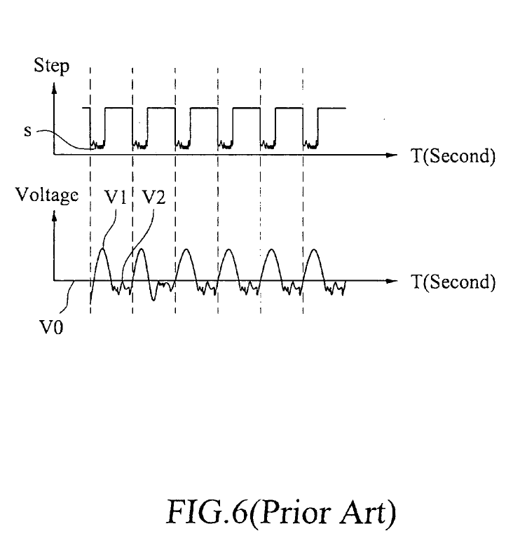

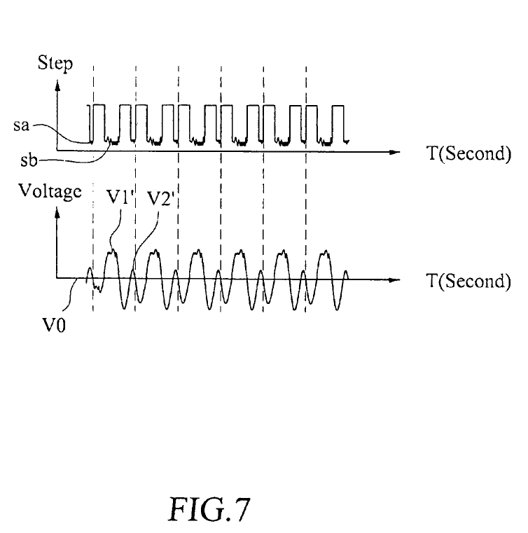

Please refer to FIGS. 6 and 7. FIG. 6 shows the output voltage of the second vibration detector 3b of FIG. 4 arranged in a horizontal direction when a user jog or walk slowly and FIG. 7 shows the output voltage of the second vibration detector 3b of FIG. 4 arranged in an angle θ to the horizontal direction when a user jog or walk slowly.

Practically, a signal is formed only when the output voltage is larger than a threshold value detectable by a control circuit. It is found that the detection of the electronic vibration detector is not sensitive enough in case that the electronic vibration detector is arranged in a horizontal direction. As it can be seen from FIG. 6, when the user swings up his hand at jogging or slow walking, the electronic vibration detector generates an output voltage V1 which is strong enough to generate a step signal s. However, when the user swings down his hand, the vibration is small and the output voltage V2 is smaller than the threshold value V0 and no vibration signal is generated. Hence, only one vibration is detected for two movements at jogging or slow walking.

Anyway, the present invention provides an improved arrangement. In the embodiment, the second electronic vibration detector 3b is mounted along an angle θ to the X direction which is parallel to the horizontal direction. It can be seen from FIG. 7 that, when the user swings up his hand at jogging or slow walking, the vibration detector generates an output voltage V1′ which is strong enough to generate a step signal sa. When the user swings down his hand, the vibration detector generates an output voltage of V2′ which is strong enough to generate a step signal sb. It is noted that the output voltage V2′ is larger than the threshold value V0. Therefore, the vibration detector is capable to count the movements precisely even at slow walking or light motion.

The present invention has been described with a preferred embodiment thereof and it is understood that many changes and modifications in the described embodiment can be carried out without departing from the scope and the spirit of the invention that is intended to be limited only by the appended claims.

What is claimed is:

1. An electronic wristwatch-type exercise signal detecting apparatus, comprising:
a watchcase being provided with a watchband at each of two opposite ends thereof to enable wearing of said watchcase on a user's wrist of one hand of the user;
a pair of electronic vibration detectors provided inside said watchcase for detecting a vibrating signal in each of two axes as the user exercises, and generating corresponding electronic signals representing said detected vibrating signals;
a vibrating signal detecting circuitry electrically connected to said electronic vibration detectors for receiving said electronic signals generated by said electronic vibration detectors and output of motion signals therefrom;
at least one electrically conductive contact area arranged at a bottom of said watchcase for contacting with a user's skin;
a pair of electrically conductive contact areas arranged at a top of said watchcase for contacting with two fingers respectively of the other hand of the user to detect the user's heartbeat/pulse signal; a pulse signal detecting circuit connected to said pair of electrically conductive contact areas at the top of said watchcase and the conductive contact areas at the bottom of said watchcase for output of a signal representing the pulse signal of the user;
a display unit provided on said watchcase; and
a control circuit including a micro-controller having inputs coupled to said vibrating signal detecting circuitry and said pulse signal detecting circuit for receiving said motion signals and said signal representing the pulse signal of the user, said micro-controller having an output coupled to said display unit for selectively displaying a numerical value representing steps of the user derived from said motion signals and a pulse rate of the user derived from said signal representing the pulse signal.

2. The electronic wristwatch-type exercise signal detecting apparatus as claimed in claim 1, wherein said control circuit includes a step signal memory electrically connected to said micro-controller for recording and storing said motion signals received by said micro-controller, and a pulse signal memory for recording and storing said signal representing the pulse signal of the user.

3. The electronic wristwatch-type exercise signal detecting apparatus as claimed in claim 1, wherein said control circuit is electrically connected to a set of operating keys.

4. The electronic wristwatch-type exercise signal detecting apparatus as claimed in claim 3, wherein said operating keys includes an on/off key, a mode-selection key, and a clear key.

5. The electronic wristwatch-type exercise signal detecting apparatus as claimed in claim 1, wherein one of said electronic vibration detectors is positioned along a direction with an angle θ to the horizontal direction, and the other electronic vibration detector is positioned along a direction with an angle θ to the vertical direction.

6. The electronic wristwatch-type exercise signal detecting apparatus as claimed in claim 6, wherein the angle θ ranges from 5 to 30 degrees.
